(19) United States
(12) Patent Application Publication
(10) Pub. No.: US 2016/0166795 A1
(43) Pub. Date: Jun. 16, 2016

(54) DEVICE, SYSTEM AND METHOD OF POSITIVE PRESSURE VENTILATION (PPV) RESUSCITATION TO CONTINUOUS POSITIVE AIRWAY PRESSURE (CPAP)

(71) Applicant: GENERAL ELECTRIC COMPANY, Schenectady, NY (US)

(72) Inventors: Harry Edward Belsinger, JR., Forest Hill, MD (US); Steven Mitchell Falk, Baltimore, MD (US); Karen P. Starr, Monkton, MD (US)

(73) Assignee: GENERAL ELECTRIC COMPANY, Schenectady, NY (US)

(21) Appl. No.: 14/571,836

(22) Filed: Dec. 16, 2014

(51) Int. Cl. A61M 16/08; A61M 16/10; A61M 16/16; A61M 16/00; A61M 16/06

(52) U.S. Cl. CPC A61M 16/0833; A61M 16/0069; A61M 16/0078; A61M 16/0666; A61M 16/16; A61M 16/1075; A61M 2240/00; A61M 2205/502; A61M 2205/52

(57) ABSTRACT

The resuscitation method and device incorporates a T-piece and/or bag and mask resuscitation device that transforms into short term (and then long term) CPAP device. This results in continuum of respiratory care in the Delivery Room, and potentially in the nursery. The respiratory support is uninterrupted. The device can include a modular design, which adds heat and humidity in a modular way, when the long term CPAP requires it. The resuscitation device and method includes a PIP phase and a PEEP phase. This is driven either manually or by a finger and gas power. The exhalation phase is PEEP, where the PEEP is a small positive pressure to ensure the lungs remain open. When the baby is spontaneously breathing, meaning no longer in need of PIP, the PEEP can be adjusted to be continuous positive pressure, or CPAP. If the CPAP is short term, there is minimal need for heating or humidifying the air. When the CPAP is used longer term, a heater and humidifier can be applied to the air source of the CPAP.

### FIELD

The present disclosure generally relates to maternal infant care. More specifically, the present disclosure relates to continuous respiratory support for an infant through respiration transformation.

### BACKGROUND

In current systems, the respiratory support of a baby has various interruptions, specifically from Positive Pressure Ventilation (PPV) to Continuous Positive Airway Pressure (CPAP). There is a need is to have this transformation uninterrupted, where the Positive End Expiratory Pressure (PEEP) from PPV is gently adjusted to a CPAP, and the baby transitions from a respiratory challenged state to non-invasive ventilation in a continuous fashion. In current systems, there is a delay of several minutes, if not hours, between resuscitation through PPV to CPAP ventilation. This transformation must be greatly reduced to no delay, or a relatively short delay.

When a baby is born, the baby may need extra resuscitative support. Perhaps the baby is not breathing, or the baby is breathing erratically, or the heart rate is really low even if the baby's breathing is normal. A resuscitation procedure is implemented and driven by actual resuscitation program guidelines and international standards. The heart rate is measured and actions are taken depending on the heart rate.

Resuscitation procedures can be effectuated by a bag and mask or a T-Piece, which is a device that is shaped like a T and is positioned on the mask, and delivers Peak Inspiratory Pressure (PIP) and PEEP to the baby and the difference between PIP and PEEP is given to the baby based on a user's finger covering a particular hole near the mask, thus gas powering instead of manual. The PIP and PEEP are gas powered into the baby and is a very episodic, transient resuscitation. The baby can be resuscitated then by alternating covering and uncovering a hole on the T-piece. This method of resuscitation is getting much more popular than the bag and mask for various safety reasons.

### SUMMARY

The resuscitation method and device incorporates a T-piece and/or bag and mask resuscitation device that transforms into short term (and then long term) CPAP device. This results in continuum of respiratory care in the Delivery Room, and potentially in the nursery. The respiratory support is uninterrupted. The device can include a modular design, which adds heat and humidity in a modular way, when the long term CPAP requires it. The resuscitation device and method includes a PIP phase and a PEEP phase. This is driven either manually or by a finger and gas power. The exhalation phase is PEEP, where the PEEP is a small positive pressure to ensure the lungs remain open. When the baby is spontaneously breathing, meaning no longer in need of PIP, the PEEP can be adjusted to be continuous positive pressure, or CPAP. If the CPAP is short term, there is minimal need for heating or humidifying the air. When the CPAP is used longer term, a heater and humidifier can be applied to the air source of the CPAP.

In one embodiment of the present application, a resuscitation system for resuscitating a patient utilizing Positive Pressure Ventilation (PPV) and transitioning to Continuous Positive Airway Pressure (CPAP), includes a flow module, wherein the flow module receives an air/O2 blend from an air/O2 supply, a resuscitation device configured to receive the air/O2 blend from the flow module to resuscitate the patient, wherein the resuscitation device includes a T-Piece, wherein the T-Piece administers the air/O₂ blend to the patient, and Peak Inspiratory Pressure (PIP) is administered to the patient in an inspiratory phase and Positive End Expiratory Pressure (PEEP) is administered to the patient in an exhalation phase, and a PEEP adjuster, wherein the PEEP adjuster is adjusted to achieve a continuous PEEP when the patient starts spontaneous breathing without removing the resuscitation device from the patient, wherein the continuous PEEP achieves a short-term Continuous Positive Airway Pressure (CPAP).

In another embodiment of the present application, a method of resuscitating a patient and transitioning to Continuous Positive Airway Pressure (CPAP) includes configuring in a resuscitation system a resuscitation device between a patient and an air/O₂ supply of the resuscitation system, resuscitating the patient with the resuscitation device with Positive Pressure Ventilation (PPV) where Positive End Expiration Pressure (PEEP) is utilized only during the exhalation phase, and adjusting the PEEP with a PEEP adjuster once the patient is spontaneously breathing to a constant positive pressure to achieve a short-term Continuous Positive Airway Pressure (CPAP) state without removing the resuscitation device from the patient.

In another embodiment of the present application, a resuscitation system for resuscitating a patient utilizing Positive Pressure Ventilation (PPV) and transitioning to Continuous Positive Airway Pressure (CPAP), includes a resuscitation device configured to receive an air/O2 blend to resuscitate the patient, wherein the resuscitation device includes a T-Piece, wherein the T-Piece administers the air/O₂ blend to the patient, and Positive End Expiratory Pressure (PEEP) is administered to the patient in an exhalation phase, and a PEEP adjuster configured on the resuscitation device, wherein the PEEP adjuster is adjusted by a user to achieve a short-term CPAP, when the patient starts spontaneous breathing, without removing the resuscitation device from the patient.

### BRIEF DESCRIPTION OF THE DRAWINGS

FIG. 1 is a schematic diagram of a system illustrating an embodiment of the present application;

FIG. 2 is a flowchart of a method illustrating an embodiment of the present application; and

FIG. 3 is a view illustrating a T-piece circuit according to an embodiment of the present application.

### DETAILED DESCRIPTION

In the present description, certain terms have been used for brevity, clearness and understanding. No unnecessary limitations are to be applied therefrom beyond the requirement of the prior art because such terms are used for descriptive purposes only and are intended to be broadly construed. The different systems and methods described herein may be used alone or in combination with other systems and methods. Various equivalents, alternatives and modifications are possible within the scope of the appended claims. Each limitation in the appended claims is intended to invoke interpretation under 35 U.S.C. §112, sixth paragraph, only if the terms “means for” or “step for” are explicitly recited in the respective limitation.

In the following detailed description, reference is made to the accompanying drawings that form a part hereof, and in which is shown by way of illustration specific embodiments that may be practiced. These embodiments are described in sufficient detail to enable those skilled in the art to practice the embodiments, and it is to be understood that other embodiments may be utilized and that logical, mechanical, electrical and other changes may be made without departing from the scope of the embodiments. The following detailed description is, therefore, not to be taken as limiting the scope of the invention.

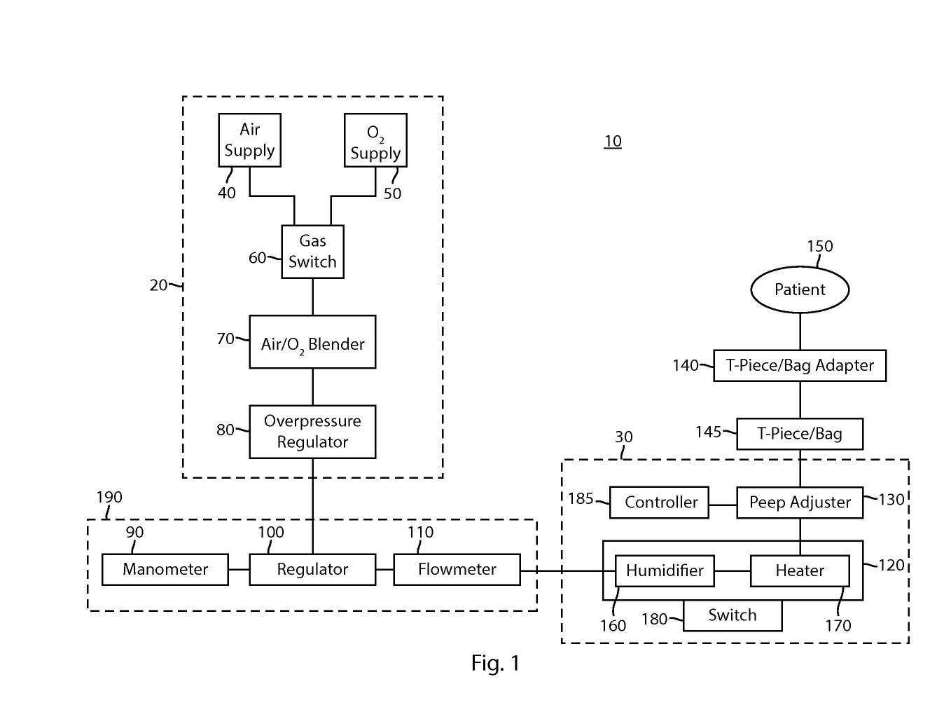

Referring first to FIG. 1, an exemplary resuscitation system 10 of the present application is illustrated. The resuscitation system 10 includes an air/oxygen (O₂) supply 20, a flow module 190, and the resuscitation device 30 of the present application. While the air/O₂ supply 20 and the flow module 190 are elements known in the art of resuscitation systems 10, the resuscitation device 30 of the present application provides a solution for the lack of continuous ventilation for a patient from resuscitation to long-term CPAP. The flow module 190 is a physical device known in the art of resuscitation systems 10 as discussed above, and includes a number of physical meters that will be described in further detail below.

Still referring to FIG. 1, an exemplary air/O₂ supply 20 will include an air supply 40, an oxygen (O₂) supply 50, a gas switch 60 that determines the source of each of the air supply 40 and O₂ supply 50 as known in the art, and passes air from the air supply 40 and O₂ from the O₂ supply 50 to an air/O₂ blender 70 that mixes the air and O₂ in an appropriate manner known in the art, and an over-pressure regulator 80 that supplies the air to the flow module 190 at the appropriate pressure.

The flow module 190 includes a manometer 90 that provides a pressure reading to a clinician utilizing the resuscitation system 10, a regulator 100 that adjusts the PIP to an appropriate level for use with the patient 150, and a flow meter 110 that adjusts the flow to the resuscitation device 30.

The resuscitation device 30 of the present application is configured to provide ventilation to a patient 150, and specifically a patient 150 that is a newborn or infant requiring resuscitation. The resuscitation device 30 may include any and all of the following items, including a T-piece or bag device 145, that when used in conjunction with a cannula or ventilation mask, provides the resuscitation PIP and PEEP to the patient 150. Items such as a cannula or ventilation mask will be referred to herein and in the claims as an accessory. Because short-term and long-term CPAP requires a cannula for appropriate and proper administration of CPAP, the T-piece or bag and mask 145 may require a T-piece/bag and mask adapter 140. While ordinarily a T-piece 145 will include a PEEP adjuster, the resuscitation device 30 may also include a PEEP adjuster 130 as well. Last, the resuscitation device 30 will also include a long-term CPAP unit 120 that includes a humidifier 160, heater 170 and switch 180. As will be described below, when transitioning the patient 150 from short-term CPAP to long-term CPAP, the long-term CPAP unit 120 will be utilized by utilizing the switch 180 to turn on the humidifier 160 and heater 170 that is a requirement of long-term CPAP. In this application, the terms “long-term CPAP” and “short-term CPAP” are utilized often. These terms are terms of the art, and are defined by what medical professionals deem to be the periods for each term.

In operation, when a patient 150 requires resuscitation, the resuscitation device 30 is utilized with the resuscitation system 10 and the patient 150 is resuscitated by utilizing a T-piece or bag and mask 145. If a T-piece 145 is utilized, then a standard mask or a cannula can be used to provide ventilation to the patient 150. Standard masks are available with existing T-piece 145 units, and likewise with a bag and mask 145 configuration. However, if a cannula is utilized, a T-piece/bag and mask adapter 140 may be utilized. During resuscitation, PIP is provided to the patient during the inspiratory phase, and PEEP is provided to the patient in the exhalation phase. As the patient 150 begins spontaneous breathing, the PEEP adjuster 130 in the resuscitation device or a PEEP adjuster included on the T-piece 145 may be adjusted by a user, such as a clinician, such that a continuous PEEP or CPAP is achieved. For short-term CPAP, the PEEP must be adjusted as described above such that there is continuous PEEP to the patient 150. However, if long-term CPAP is desired, then the long-term CPAP unit 120 is switched on with the switch 180 so that the humidifier 160 and heater 170 are activated.

The resuscitation device 30 also includes a controller 185. The controller 185 includes a processor, storage system, software, communication interface and a user interface (none shown). The controller 185 may load and execute software from the storage system, including a software module. When executed by the controller 185, the software directs the processor to operate as described herein in further detail. Specifically, the controller may control the adjustment of the PEEP, given the breathing parameters of the patient, and the appropriate PEEP levels for long-term and short-term CPAP. This ability for the PEEP to be adjusted by executed software within the controller 185 improves the function of the system 10 by allowing the resuscitation device 30 to guide the patient from PPV resuscitation to short-term, and then long-term CPAP.

Although the controller 185 as depicted in FIG. 1 includes one software module in the present example, it should be understood that one or more modules could provide the same operation. Similarly, while the description as provided herein refers to a controller 185 and a processor, it is to be recognized that implementations of such systems can be performed using one or more processors, which may be communicatively connected, and such implementations are considered to be within the scope of the description.

The processor can comprise a microprocessor and other circuitry that retrieves and executes software from storage system. The processor can be implemented within a single processing device but can also be distributed across multiple processing devices or sub-systems that cooperate in executing program instructions. Examples of processors include general-purpose central processing units, application specific processors, and logic devices, as well as any other type of processing device, combinations of processing devices, or variations thereof.

The storage system can comprise any storage medium readable by processor, and capable of storing software. The storage system can include volatile and nonvolatile, removable and non-removable media implemented in any method or technology for storage of information, such as computer readable instructions, data structures, program modules, or other data. Storage system 204 can be implemented as a single storage device but may also be implemented across multiple storage devices or subsystems. Storage system can further include additional elements, such as a controller capable of communicating with the processor.

Examples of storage media include a random access memory, read only memory, magnetic disks, optical disks, flash memory, virtual memory, and non-virtual memory, magnetic sets, magnetic tape, magnetic disk storage or other magnetic storage devices, or any other medium which can be used to store the desired information and that may be accessed by an instruction execution system, as well as any combination or variation thereof, or any other type of storage medium.

User interface can include a mouse, a keyboard, a voice input device, a touch input device for receiving a gesture from a user, a motion input device for detecting non-touch gestures and other motions by a user, and other comparable input devices and associated processing elements capable of receiving user input from a user. Output devices such as a video display or graphical display can display and interface further associated with embodiments of the system and method as disclosed herein. The speakers, printers, haptic devices, and other types of output devices may also be included in the user interface.

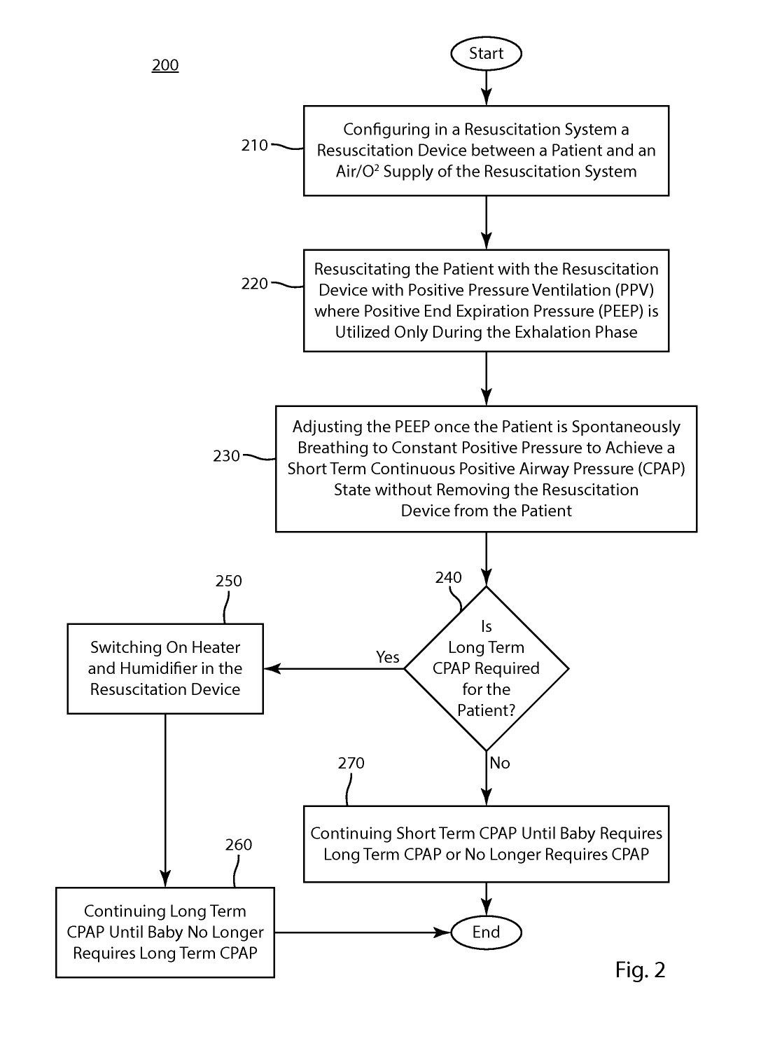

Referring now to FIG. 2, a method 200 of the present application is illustrated in a flow chart. In step 210, the resuscitation system (FIGS. 1, 10) is configured with the resuscitation device between a patient and an air/O2 supply of the resuscitation system. As discussed previously with respect to FIG. 1, a resuscitation device 30 is implemented into the resuscitation system 10 so that it may be connected to the patient 150 being resuscitated. In step 220, the patient 150 is resuscitated with the resuscitation device 30 utilizing PPV, where PEEP is utilized only during the exhalation phase, while PIP is utilized during the inspiratory phase of the patient 150 breathing. In step 230, the PEEP is adjusted manually by the clinician or automatically by the system 10 and/or system software once the patient 150 is spontaneously breathing to constant positive pressure to achieve a short-term CPAP state without removing the resuscitation device 30 from the patient 150. As noted previously, if the resuscitation device 30 is utilizing a bag and mask, the bag and mask will preferably stay connected to the patient during the transition from PPV to CPAP. This will also be the case when utilizing a T-piece having a mask. If the clinician decides to switch from a T-piece and face mask 145 to a T-piece 145 having an adapter 140 and a cannula, then there will be an instantaneous time period when the resuscitation mask 145 is removed, and the Cannula is inserted into the patient 150 and connected to the resuscitation device 30. If long-term CPAP is required for the patient 150 in 240, then in step 250, a heater 170 and humidifier 160 in the resuscitation device 30 is switched on by the clinician, and if long-term CPAP is not required for this particular patient 150 in 240, then in step 270 the short-term CPAP continues until the patient 150 requires long-term CPAP or no longer requires CPAP at all. In step 260, likewise long-term CPAP is continued until the patient 150 no longer requires long-term CPAP.

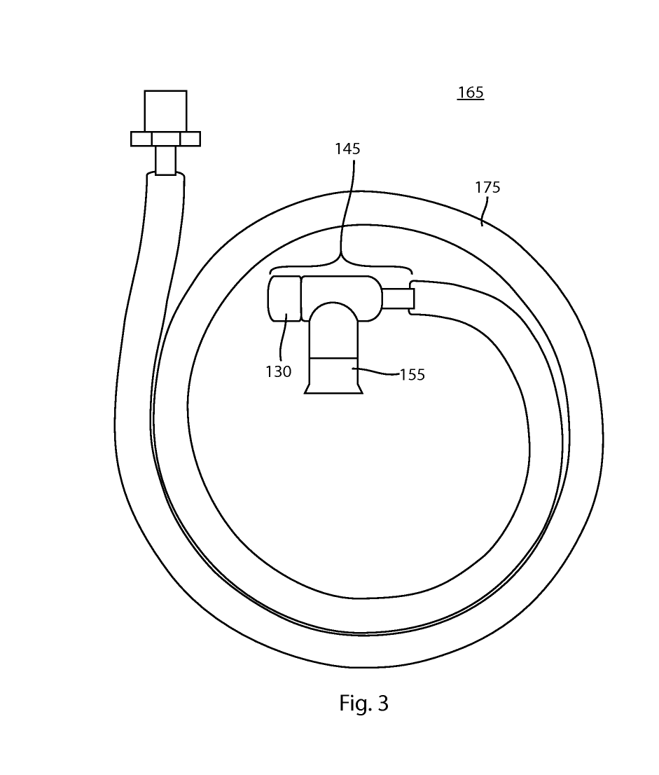

Referring now to FIG. 3, a T-piece circuit 165 of the present application is illustrated. Here, the T-piece circuit 165 includes a T-piece conduit 175 and the T-piece 145. As illustrated, the T-piece 145 includes a T-piece endcap 155 that may be removed such that a T-piece adapter 140 (not shown) or a mask (not shown) may be fitted to this portion of the T-piece 145 for delivery of ventilation to the patient 150. In one embodiment, the T-piece 145 also includes a PEEP adjuster 130, as explained previously. The end of the T-piece conduit 175 opposite of the T-piece 145 is connected to the resuscitation device 30. While it is not explicitly shown in this drawing, the T-piece 145 may include an adapter 140 that allows the T-piece 145 to connect to a common cannula (not shown) to provide long-term CPAP to the patient 150.

In the foregoing description, certain terms have been used for brevity, clearness, and understanding. No unnecessary limitations are to be inferred therefrom beyond the requirement of the prior art because such terms are used for descriptive purposes and are intended to be broadly construed. The different configurations, systems, and method steps described herein may be used alone or in combination with other configurations, systems and method steps. It is to be expected that various equivalents, alternatives and modifications are possible within the scope of the appended claims.

I claim:

1. A resuscitation system for resuscitating a patient utilizing Positive Pressure Ventilation (PPV) and transitioning to Continuous Positive Airway Pressure (CPAP), the system comprising:
a flow module, wherein the flow module receives an air/O2 blend from an air/O2 supply;
a resuscitation device configured to receive the air/O2 blend from the flow module to resuscitate the patient, wherein the resuscitation device includes a T-Piece, wherein the T-Piece administers the air/O₂ blend to the patient, and Peak Inspiratory Pressure (PIP) is administered to the patient in an inspiratory phase and Positive End Expiratory Pressure (PEEP) is administered to the patient in an exhalation phase; and
a PEEP adjuster, wherein the PEEP adjuster is adjusted to achieve a continuous PEEP when the patient starts spontaneous breathing without removing the resuscitation device from the patient, wherein the continuous PEEP achieves a short-term Continuous Positive Airway Pressure (CPAP).

2. The system according to claim 1, further comprising a long-term CPAP unit including a heater and a humidifier, such that the long-term CPAP unit is activated when the patient needs to achieve a long-term CPAP, and the air/O₂ blend is heated and humidified by the long-term CPAP unit when the long-term CPAP unit is activated.

3. The system according to claim 2, wherein the long-term CPAP unit is configured in the resuscitation device and is activated by a switch configured with the resuscitation device.

4. The system according to claim 1, wherein the PEEP adjuster is configured on the T-Piece.

5. The system according to claim 1, wherein the PEEP adjuster is configured on the resuscitation device.

6. The system according to claim 1, wherein the flow module includes a manometer, a regulator and a flowmeter, and further wherein the regulator controls the PIP to the patient.

7. The system according to claim 1, wherein the PEEP is adjusted by a user.

8. The system according to claim 1, wherein a set of code is executed by a processor and the PEEP is adjusted by directions provided by the set of executed code.

9. The system according to claim 1, wherein the resuscitation device includes a bag and mask, wherein the bag and mask administers the air/O₂ blend to the patient.

10. The system according to claim 9, wherein the resuscitation device includes a bag and mask adapter such that a breathing accessory may be coupled with the bag and mask.

11. A method of resuscitating a patient and transitioning to Continuous Positive Airway Pressure (CPAP), the method comprising:
configuring in a resuscitation system a resuscitation device between a patient and an air/O₂ supply of the resuscitation system;
resuscitating the patient with the resuscitation device with Positive Pressure Ventilation (PPV) where Positive End Expiration Pressure (PEEP) is utilized only during the exhalation phase; and
adjusting the PEEP with a PEEP adjuster once the patient is spontaneously breathing to a constant positive pressure to achieve a short-term Continuous Positive Airway Pressure (CPAP) state without removing the resuscitation device from the patient.

12. The method according to claim 11, further comprising activating a long-term CPAP unit including a heater and a humidifier, such that the long-term CPAP unit is activated when the patient needs to achieve a long-term CPAP, and the air/O₂ blend is heated and humidified by the long-term CPAP unit when the long-term CPAP unit is activated.

13. The method according to claim 12, wherein the long-term CPAP unit is configured in the resuscitation device and is activated by a switch configured with the resuscitation device.

14. The method according to claim 11, wherein the PEEP adjuster is configured on the T-Piece.

15. The method according to claim 11, wherein the PEEP adjuster is configured on the resuscitation device.

16. The method according to claim 11, further comprising a user adjusting the PEEP.

17. The method according to claim 11, further comprising executing a set of code with a processor and adjusting the PEEP with directions provided by the set of executed code.

18. The method according to claim 11, administering the air/CO₂ blend to the patient with a bag and mask.

19. The method according to claim 18, wherein the resuscitation device includes a bag and mask adapter such that a breathing accessory may be coupled with the bag and mask.

20. A resuscitation system for resuscitating a patient utilizing Positive Pressure Ventilation (PPV) and transitioning to Continuous Positive Airway Pressure (CPAP), the system comprising:
a resuscitation device configured to receive an air/O2 blend to resuscitate the patient, wherein the resuscitation device includes a T-Piece, wherein the T-Piece administers the air/O₂ blend to the patient, and Positive End Expiratory Pressure (PEEP) is administered to the patient in an exhalation phase; and
a PEEP adjuster configured on the resuscitation device, wherein the PEEP adjuster is adjusted by a user to achieve a short-term CPAP, when the patient starts spontaneous breathing, without removing the resuscitation device from the patient.
